(19) United States
(12) Patent Application Publication
(10) Pub. No.: US 2026/0199028 A1
(43) Pub. Date: Jul. 16, 2026

(54) SYSTEM AND METHOD FOR USING MACHINE LEARNING TO PROVIDE NAVIGATION GUIDANCE AND RECOMMENDATIONS RELATED TO REVISION SURGERY

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Nathan ZAMARRIPA, Kittery Point, ME (US); Alyssa ANDRADE, Dedham, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/563,114

(22) Filed: Mar. 11, 2026

Related U.S. Application Data
(63) Continuation of application No. PCT/US2024/038949, filed on Jul. 22, 2024.
(60) Provisional application No. 63/585,372, filed on Sep. 26, 2023.

(51) Int. Cl. A61B 34/20; G16H 20/40; G16H 40/67; A61B 90/00

(52) U.S. Cl. CPC A61B 34/20; G16H 20/40; G16H 40/67; A61B 2034/2065; A61B 2090/364

(57) ABSTRACT

In some examples, a computer-implemented method may include receiving one or more images related to a surgical site of a patient. The method may include identifying, based on the one or more images and using one or more trained machine learning models, one or more pieces of hardware installed at the surgical site. The method may include determining, based on the one or more pieces of hardware and using the one or more machine learning models, navigation guidance to uninstall the one or more pieces of hardware during revision surgery. The method may include transmitting the navigation guidance to a computing device associated with a surgeon to perform the revision surgery.

### CROSS-REFERENCE TO RELATED APPLICATIONS

This application claims the benefit of PCT Application No. PCT/US2024/038949, filed July 22, 2024, and U.S. Provisional Application No. 63/585,372, filed September 26, 2023, titled “System and Method for Using Machine Learning to Provide Navigation Guidance and Recommendations Related to Revision Surgery.” These applications are incorporated herein by reference as if reproduced in full below.

### BACKGROUND

The anterior cruciate ligament (ACL) serves as the primary mechanical restraint in the knee to resist anterior translation of the tibia relative to the femur. Similarly the posterior cruciate ligament (PCL) serves as a mechanical restraint to resist posterior translation of the tibia relative to the femur. These cruciate ligaments contribute significantly to knee stability, and ACL injury is quite common. Most ACL injuries are complete tears of the ligament.

As ACL injuries occur often in patients that are young and active, reconstruction of the ACL is performed to enable return to activity. The goal is to restore stability of the knee and reduce the chances of further damage to the meniscus and articular cartilage that may lead to degenerative osteoarthritis. Reconstruction may consist of placement of a substitute graft (e.g., autograft from either the central third of the patellar tendon or the hamstring tendons). The ends of the graft are placed into respective tunnels prepared through the femur and the tibia. The ends of the graft may be attached using interference screws or a suspensory fixation device like the ENDOBUTTON™ brand fixation devices manufactured by Smith & Nephew of Andover, Massachusetts, USA.

In some cases, ACL reconstruction surgery may fail and revision surgery may need to be performed. Revision ACL surgeries may be challenging for several reasons. First, the surgeon performing the revision may not have been the surgeon who performed the primary ACL repair. Additionally, tunnel placement, tunnel width, and/or the hardware manufacturer, among other things, may be unknowns during preoperative planning.

### SUMMARY

In some examples, a computer-implemented method may include receiving one or more images related to a surgical site of a patient. The method may include identifying, based on the one or more images and using one or more trained machine learning models, one or more pieces of hardware installed at the surgical site. The method may include determining, based on the one or more pieces of hardware and using the one or more machine learning models, navigation guidance to uninstall the one or more pieces of hardware during revision surgery. The method may include transmitting the navigation guidance to a computing device associated with a surgeon to perform the revision surgery.

In some examples, a system includes a memory storing instructions and a processing device communicatively coupled to the memory. The processing device executes the instructions to receive one or more images related to a surgical site of a patient, identify, based on the one or more images and using one or more trained machine learning models, one or more pieces of hardware installed at the surgical site, determine, based on the one or more pieces of hardware and using the one or more machine learning models, navigation guidance to uninstall the one or more pieces of hardware during revision surgery, and transmit the navigation guidance to a computing device associated with a surgeon to perform the revision surgery.

In some examples, a tangible, non-transitory computer-readable storage medium stores instructions that, when executed, cause a processing device to receive one or more images related to a surgical site of a patient, identify, based on the one or more images and using one or more trained machine learning models, one or more pieces of hardware installed at the surgical site, determine, based on the one or more pieces of hardware and using the one or more machine learning models, navigation guidance to uninstall the one or more pieces of hardware during revision surgery, and transmit the navigation guidance to a computing device associated with a surgeon to perform the revision surgery.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows a conceptual drawing of a surgical site with various objects within the surgical site tracked, in accordance with at least some embodiments;

FIG. 6 is an example video display showing portions of a femur and having visible therein a bone fiducial, in accordance with at least some embodiments;

FIG. 7 is an example video display showing intraoperative changes to a tunnel path, in accordance with at least some embodiments;

FIG. 8 shows a computing device executing an artificial intelligence engine, in accordance with at least some embodiments;

FIG. 9 is an example flow chart of a method for using machine learning models to provide navigation guidance for revision surgery, in accordance with at least some embodiments;

FIG. 10 shows a computer system in accordance with at least some embodiments.

### DEFINITIONS

Various terms are used to refer to particular system components. Different companies may refer to a component by different names – this document does not intend to distinguish between components that differ in name but not function. In the following discussion and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counter bore may thus define an internal shoulder. However, the term “counterbore” shall not be read to imply any method of creation. A counterbore may be created in any suitable way, such as drilling, boring, laser drilling, or casting.

The terms “input” and “output” when used as nouns refer to connections (e.g., electrical, software), and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections. In systems implemented in software, these “inputs” and “outputs” define parameters read by or written by, respectively, the instructions implementing the function.

“Controller” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), or a field programmable gate array (FPGA), configured to read inputs and drive outputs responsive to the inputs.

“Real-time” may refer to an action, operation, method, function, instruction, etc. being performed within 2 seconds. “Near real-time” may refer to an action, operation, method, function, instruction, etc. being performed between 2 seconds and 1 minute.

The term “supervised learning,” when used in a machine learning context, may refer to a technique that uses labeled datasets to train algorithms to classify data or predict outcomes accurately. Labeled input data may be provided to a machine learning model that adjusts its weights and/or other parameters until the machine learning model is trained to properly identify labeled outputs. The algorithm measures its accuracy through a loss function by adjusting the weights and/or parameters until an error satisfies a threshold level.

The term “unsupervised learning,” when used in a machine learning context, may refer to a technique that analyzes and clusters unlabeled datasets by identifying patterns or data groupings in the datasets based on similarities and/or differences among the datasets. One example of unsupervised learning includes clustering. Clustering may refer to a data mining technique that groups unlabeled data based on the similarities or differences within different parts of the unlabeled data. Another example of unsupervised learning comprises association rules. Association rules may refer to a rule-based method for finding relationships between variables in a given dataset. Another example of unsupervised learning comprises dimensionality reduction. Dimensionality reduction may refer to a technique used when the number of features, or dimensions, in a dataset is too high. Dimensionality reduction reduces the number of data inputs to a manageable size while maintaining the integrity of the dataset.

The term “reinforcement learning,” when used in a machine learning context, may refer to a technique that enables an agent to learn in an interactive environment by trial and error by using feedback from its own actions and experiences. Reinforcement learning uses rewards and punishments as signals to indicate, during the training phase of a machine learning model, positive and negative behaviors of the agent. A goal of reinforcement learning is to discover a suitable machine learning model that maximizes the total cumulative reward of or associated with the agent.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems for using machine learning models to provide navigation guidance for performing revision surgery based on identified hardware installed in a portion of a patient’s body. Identifying the existing hardware inside the patient is a complex and time-consuming aspect of a preoperative surgery plan. Conventionally, if the hardware is not correctly identified preoperatively, the result may be a rescheduled or delayed revision surgery. Consequently, there is a clinical need for a digital solution that can accurately identify the existing hardware and enable a surgeon to obtain the relevant drivers and instruments and tools ahead of revision surgery. The disclosed embodiments provide the desired solution. There exist a vast number of metallic and nonmetallic absorbable and non-resorbable screws on the market and each requires the correct driver / tool to remove it. A sample list of custom drivers includes hexagonal, cannulated, quadrangular, tri-lobe, torx – all in various metric and imperial sizes. The disclosed embodiments used trained machine learning models to analyze images (e.g., computed tomography images) to identify installed hardware (e.g., screws) and output the tools to use to uninstall the hardware and navigation guidance to perform revision surgery. Further, the machine learning models may output one or more probabilities related to a success rate of performing revision surgery and/or colliding with installed hardware during the revision surgery.

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Thus, the description and developmental context shall not be read as a limitation of the applicability of the teachings. In order to orient the reader, the specification first turns a description of the knee.

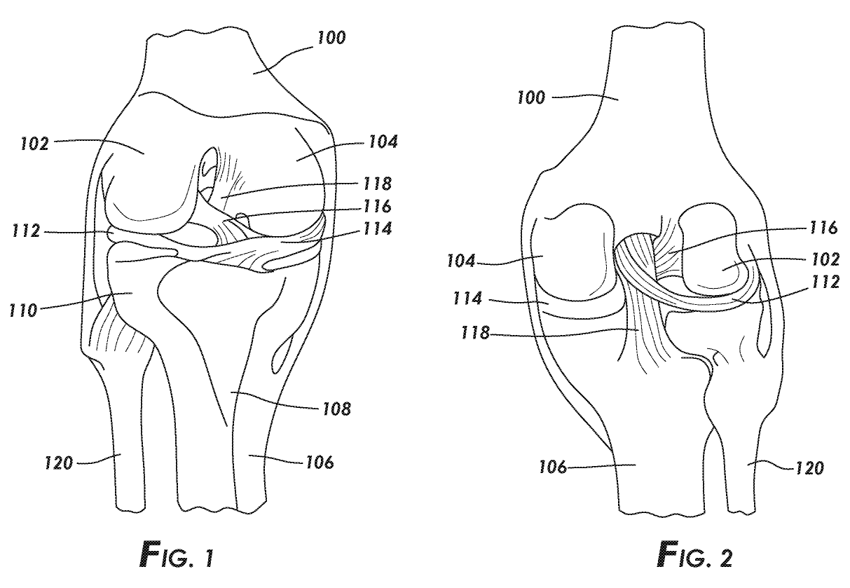

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy’s tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

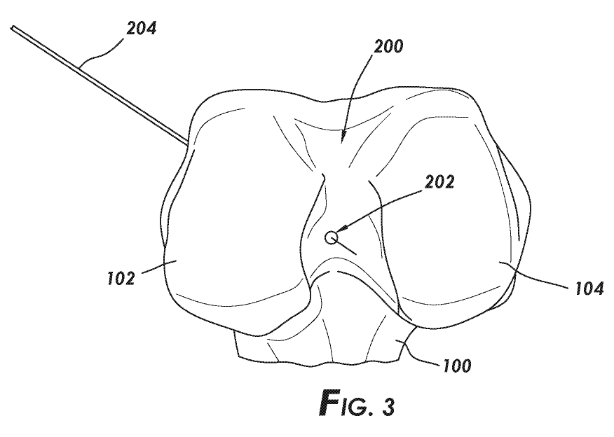

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon’s selection for location of the port through the skin, and through which the various instruments are inserted into the knee, it may be difficult to reach the attachment location of the native ACL to the femur 100.

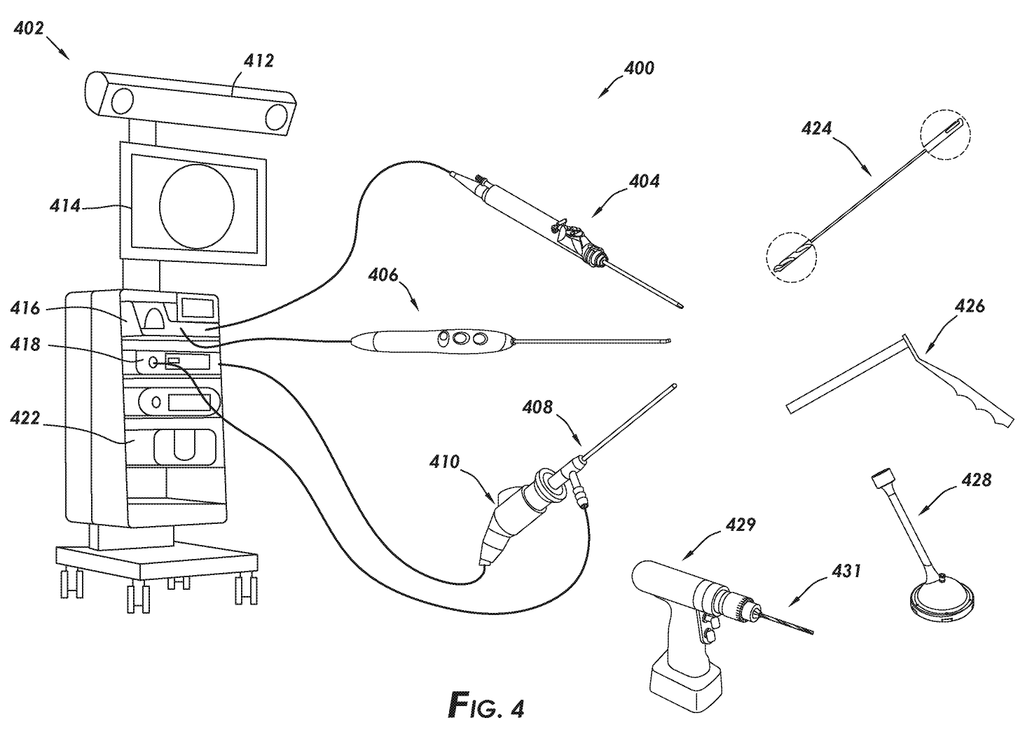

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define inside aperture 202 within the femoral notch 200, the inside aperture 202 closer to the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the drill wire 204 is closely aligned with a planned-tunnel path, the femoral tunnel is created by boring or reaming with another instrument (e.g., a reamer) that may use the drill wire 204 as a guide. In some cases, a socket or counterbore is created on the intercondylar notch side to accommodate the width of the graft that extends into the bone, and that counterbore may also be created using another instrument (e.g., reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The further examples discussed below are thus based on an inside-out procedure, but such should not be read as a limitation. The specification now turns to an example surgical system.

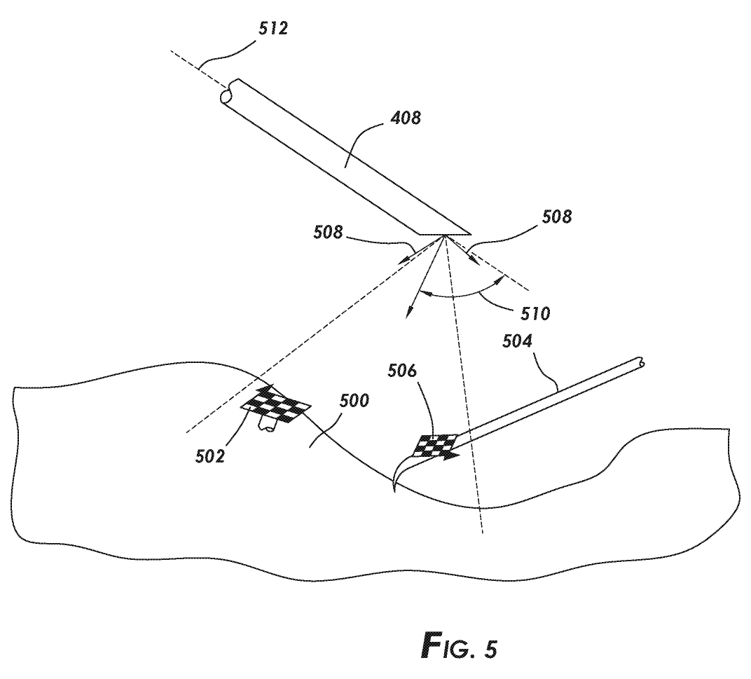

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 not only provides light to the arthroscope 408 and displays images received from the camera head 410, but also implements various additional aspects, such as displaying a single aimer indicator that changes aspects depending on when a tool is in coaxial alignment, displaying an overlay of iso-angles representing knee angles to achieve a target exit angle, and so forth. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 may then assess whether the pilot tunnel matches or closes matches the planned-tunnel path. If the pilot tunnel is deemed sufficient, then the drill wire 424 may be used as a guide for creating the full-diameter throughbore for the tunnel, or alternatively, for creating a counterbore stemming from the intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As will be discussed in greater detail below, the calibration assembly 428 may be used to detect optical distortion in images received by the surgical controller 418 through the arthroscope 408 and attached camera head 410.

Additional tools and instruments will be present, such as a drill 429 for drilling with the drill wire 424, various reamers 431 for creating the throughbore and counterbore aspects of the tunnel, and various tools for suturing and anchoring the graft in place. Some of these additional tools and instruments are not shown so as not to further complicate the figure.

The specification now turns to a workflow for an example ACL repair. The workflow may be conceptually divided into a preoperative planning and intraoperative repair. The intraoperative repair workflow may be further conceptually divided into optical system calibration, model registration, intraoperative tunnel-path planning, intraoperative and tunnel creation. Each will be addressed in turn.

### PREOPERATIVE PLANNING

In accordance with various examples, an ACL repair starts with preoperative imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The discussion that follows assumes MRI imaging, but again many different types of preoperative imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of ACL repair and specifically selecting a tunnel path through the femur, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native ACL to the femur within the femoral notch. In some cases, selecting the entry location within the femoral notch may involve use of a Bernard & Hertel Quadrant or grid placed on a fluoroscopic image, or placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the preoperative planning may comprise: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia.

In some embodiments, using the three-dimensional bone model, the surgeon may select a planned value for a socket or counterbore depth of a reamer into a bone. The surgeon may select the socket or counterbore depth such that a socket is not created that is too long, thereby resulting in a thin bone wall that may be too close to the cortex. A bone wall that is thin and too close to the cortex may result in a failure of the ACL reconstruction. In addition, using the three-dimensional bone model, the surgeon may select a tunnel throughbore diameter, a counterbore diameter, and the like. One or more reamers may be selected based on the diameters of the tunnel throughbore diameter and/or the counterbore diameter.

In some embodiments, the three-dimensional bone model may include a virtual representation of the imaged bones of the patient mapped into a three-dimensional coordinate space (e.g., virtual space). The dimensions, size, density, length, width, depth, height, shape, position, configuration, orientation, and the like of the bones may be accurately represented in the three-dimensional coordinate space.

Other surgical parameters may also be selected during the preoperative planning, such as desired post-repair flexion, and the like, but those additional surgical parameters are omitted so as not to unduly complicate the specification.

### INTRAOPERATIVE REPAIR

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient’s skin, and in fact before the patient is wheeled into the surgical room. Nevertheless, such steps and procedures are considered intraoperative as they take place in the surgical setting and with the surgical equipment and instruments used to perform the actual repair.

The example ACL repair is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the bone within the three-dimensional coordinate space of the view of the arthroscope, the location of the reamer within the three-dimensional coordinate space of the view of the arthroscope, and location of the various instruments (e.g., the drill wire 424, the aimer 426) within the three-dimensional coordinate space of the view of the arthroscope. The specification turns to brief description of such tracking techniques.

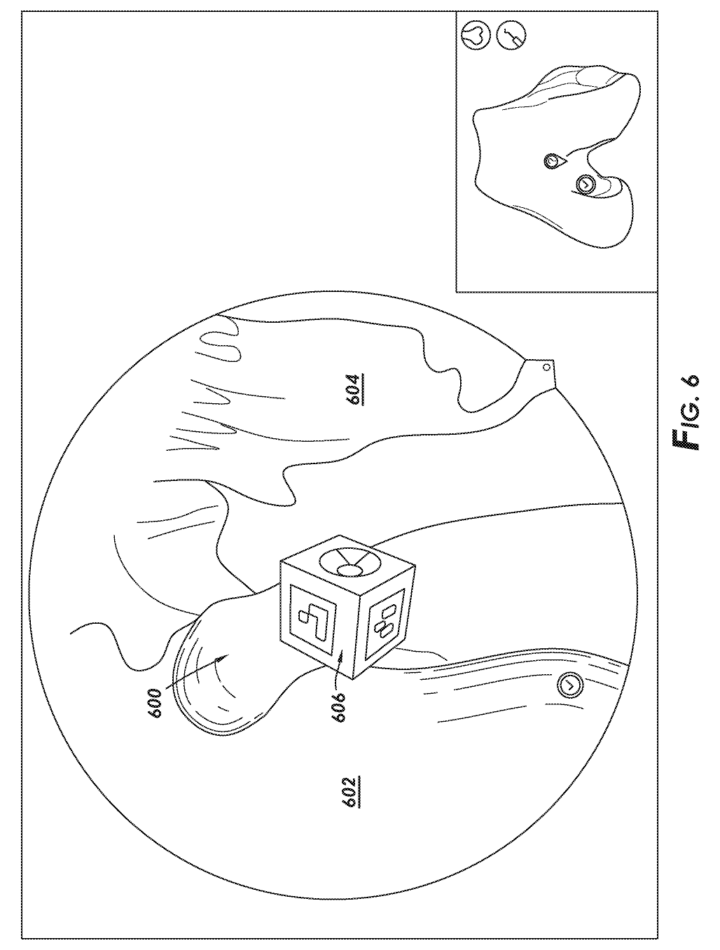

FIG. 5 shows a conceptual drawing of a surgical site with various objects within the surgical site. In particular, visible in FIG. 5 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light. In the example of FIG. 5, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410 (FIG. 4). The images detected by the capture array within the camera head 410 are sent electronically to the surgical controller 418 (FIG. 4) and displayed on the display device 414 (FIG. 4). In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30^{o} arthroscope the viewing direction forms a 30^{o} angle to the longitudinal central axis of the arthroscope, the angle measured as an obtuse angle beyond the distal end of the arthroscope. In many cases for ACL repair, the surgeon selects a 30^{o} arthroscope or a 45^{o} arthroscope based on location the port created through the skin of the patient. In the example of FIG. 5, the view angle 510 of the arthroscope 408 forms a non-zero angle to the longitudinal central axis 512 of the arthroscope 408.

Still referring to FIG. 5, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410 (FIG. 4).

The probe fiducial 506 is shown as a planar element attached to the touch probe 504. The touch probe 504 may be used, as discussed more below, to “paint” the surface of the bone 500 as part of the registration of the bone 500 to the three-dimensional bone model, and the touch probe 504 may also be used to indicate revised-tunnel entry locations in the case of intraoperative changes to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the point of the touch probe 504, may be determined from images captured by the arthroscope 408 and attached camera head 410 (FIG. 4).

Other instruments within the view of the arthroscope 408 may also have fiducials, such as the drill wire 424 (FIG. 4) and aimer 426 (FIG. 4), but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426 (FIG. 4)), the location may be tracked by an optical array coupled to the aimer and viewed through the camera 412 (FIG. 4), such as a stereoscopic camera. The location within the three-dimensional coordinate space of the camera 412 is then transformed into the three-dimensional coordinate space of the view of the example arthroscope to determine location of the distal end within the surgical site.

The images captures by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process.

### OPTICAL SYSTEM CALIBRATION

In example systems, prior to use within each surgical procedure, the endoscopic optical system is calibrated to account for the various optical distortions. In particular, various embodiments comprise a system for calibrating the endoscopic optical system. Referring again to FIG. 4, the example system comprises the surgical controller 418, the arthroscope 408, the camera head 410, and the calibration assembly 428. In particular, the calibration may comprise placing the arthroscope 408 into the calibration assembly 428. The calibration assembly 428 holds the arthroscope 408 in a fixed relationship to a calibration target on an inside surface of the calibration assembly 428. Once the distal end of the arthroscope 408 is within the calibration assembly 428, the example method comprises capturing a plurality of images of the calibration target, with each image captured at a unique rotational relationship between the camera head 410 and the arthroscope 408, with the unique rotational relationships relative to a longitudinal central axis of the arthroscope 408. Using the plurality of images, the example surgical controller 418 creates a characterization function that characterizes optical distortion between the calibration target and the capture array within the camera head 410. The characterization function may include a calibration for determining orientation of fiducial markers visible within the surgical site (e.g., bone fiducial 502, probe fiducial 506) by way of the arthroscope 408 and attached camera head 410.

### MODEL REGISTRATION AND HUMAN-IN-THE-LOOP REGISTRATION VERIFICATION

The next example step in the intraoperative procedure is the registration of the bone model(s) created during the preoperative planning. That is, during the preoperative planning stage, preoperative imaging (e.g., MRI) of the knee takes place, including the relevant anatomy like the lower portion of the femur, the upper portion of the tibia, and the articular cartilage. The preoperative imaging can be segmented such that a volumetric model or three-dimensional model of the anatomy is created. More specifically to the example of ACL repair, and specifically selecting a tunnel path through the femur, a three-dimensional bone model of the lower portion of the femur is created during the preoperative planning. Further, a depth of the tunnel path may be selected and a diameter of the tunnel may be selected. The depth and diameter may be represented in the volumetric or three-dimensional model (e.g., a virtual space including axes and coordinates (e.g., X, Y, Z)).

During the intraoperative repair, the three-dimensional bone models are provided to the surgical controller 418. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient’s skin, the surgical controller 418 also receives video images of the femur. In order to relate the three-dimensional bone model to the images received by way of the arthroscope 408 and camera head 410, the surgical controller 418 registers the three-dimensional bone model to the images of the femur received by way of the arthroscope 408 and camera head 410.

In accordance with example methods, a fiducial marker or bone fiducial (e.g., bone fiducial 502 of FIG. 5) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial is placed within the intercondylar notch superior to or above the expected location of the tunnel through lateral condyle.

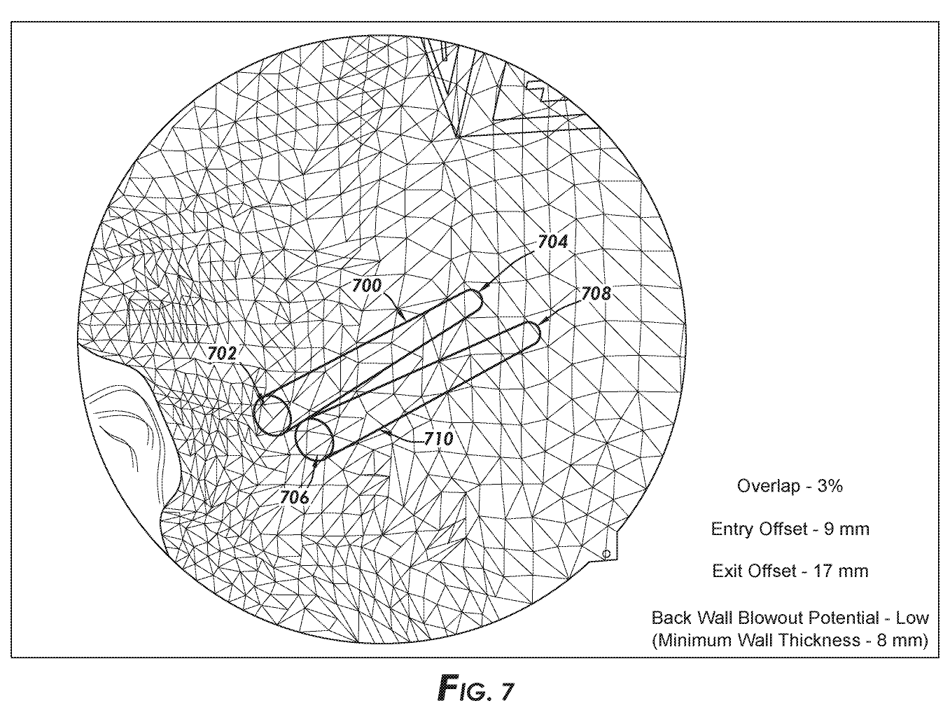

FIG. 6 is an example video display showing portions of a femur and a bone fiducial. The display may be shown, for example, on the display device 414 (FIG. 4) associated with the device cart 402 (FIG. 4), or any other suitable location. In particular, visible in FIG. 6 is a femoral notch or intercondylar notch 600, a portion of the lateral condyle 602, a portion of the medial condyle 604, and an example bone fiducial 606. The bone fiducial 606 is a fiducial comprising a cube member. Of the six outer faces of the cube member, the bottom face is associated with an attachment feature (e.g., a screw). The bottom face will be close to or abut the bone when the bone fiducial 606 is secured in place, and thus will not be visible in the view of the arthroscope 408 (FIG. 4). The outer face opposite the bottom face includes a placement feature used to hold the bone fiducial 606 prior to placement, and to attach the bone fiducial 606 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 606 represents a fixed location on the outer surface of the bone in the view of the arthroscope 408, even as the position of the arthroscope 408 is moved and changed relative to the bone fiducial 606. Initially, the location of the bone fiducial 606 with respect to the three-dimensional bone model is not known to the surgical controller 418, hence the need for the registration of the three-dimensional bone model.

In or order to relate or register the bone visible in the video images to the three-dimensional bone model, the surgical controller 418 (FIG. 4) is provided and thus receives a plurality of locations of an outer surface of the bone. For example, the surgeon may touch a plurality of locations using the touch probe 504 (FIG. 5). As previously discussed, the touch probe 504 comprises a probe fiducial 506 (FIG. 5) visible in the video images captured by the arthroscope 408 (FIG. 4) and camera head 410 (FIG. 4). The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” locations of the outer surface of the bone relative to the bone fiducial 606. Given that the touch probe 504 is a relatively inflexible instrument, in other examples the tracking of the touch probe 504 may be by optical tracking of an optically-reflective array outside the surgical site (e.g., tracking by the camera 412 (FIG. 4)) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone. In some embodiments, other modes of capturing the shape of the bone relative to the bone fiducial 606 may include using computed tomography (CT) and/or fluoroscopy with or without contrast (e.g., a substance that is injected into an intravenous line or taken by mouth to enable the body part (e.g., bone tissue and structure) being examined to be seen more clearly). CT scans may use an X-ray beam that traverses a body part externally in a circular motion to obtain many different views of the same body part and those images may be used to generate a two-dimensional form of the body part on a video display. Fluoroscopy may refer to imaging technique that uses a fluoroscope to obtain real-time moving images of a body part of the patient. CT and/or fluoroscopy may enable obtaining updated images of the outer surface of the bone relative to the bone fiducial 606. In some embodiments, another mode of capturing the shape of the bone relative to the bone fiducial 606 may include using an externally tracked ultrasound probe. An external tracker may be used to identify a position and orientation of images obtained by an ultrasound probe that may be merged to identify the outer surface of the bone relative to the bone fiducial 606.

A video display may be displayed during a registration procedure showing portions of a femur, a bone fiducial, and an overlaid representation of the three-dimensional bone model. The display may be shown, for example, on the display device 414 (FIG. 4) associated with the device cart 402 (FIG. 4), or any other suitable location. In order to address potential invalid registrations between the three-dimensional bone model and the bone visible in the images, in example methods and systems an initial registration between the bone visible in the video images and the three-dimensional bone model is verified using a human-in-the-loop process. More particularly, in example cases, after receiving the plurality of locations of an outer surface of the bone visible in the images and performing an initial registration to the three-dimensional bone model, the surgical controller 418 displays a representation of the three-dimensional bone model overlaid on portions of the bone visible in the video images, the displaying and overlaying on the display device 414, or other suitable location. That is, the example surgical controller 418 overlays a representation of the three-dimensional bone model (e.g., by way of a polygon mesh, or mesh model) on the display of the images captured by the arthroscope 408 and camera head 410, wherein the rotational and translational alignment of the three-dimensional bone model is correlated to the bone visible in the video images as anchored by the bone fiducial 606.

The surgeon, in turn, visually studies the overlaid representation of the three-dimensional bone model relative to the underling bone visible in the video images to determine whether the registration process was correct. More particularly, the surgeon visually compares the overlaid representation of the three-dimensional bone model to the portion of the bone visible in the video images to determine whether the three-dimensional bone model sufficiently matches the bone visible in the video images. Much like the registration process itself, the human-in-the-loop verification of registration is a non-deterministic exercise. Slight variances between the three-dimensional bone model and the bone visible in the video images may be tolerated, yet nevertheless the registration process may be considered correct in the sense that the three-dimensional bone model may be reliably used to help guide placement of the tunnel path (e.g., here the femoral tunnel path), or assist the surgeon in intraoperative changes to the planned-tunnel path. In such cases, the surgeon may provide the surgical controller 418, and the surgical controller 418 may thus receive, an indication that the three-dimensional bone model is correctly registered to the bone visible in the video images.

On the other hand, if the overlay of the three-dimensional bone model shows misalignment with the bone visible in the video images, the surgeon may elect to restart the registration process, such as by providing to the surgical controller 418, and the surgical controller 418 receiving again, a plurality of locations of the outer surface of the bone. The surgical controller 418 may then perform anew the registration procedure. In other cases, the surgeon may elect to provide additional locations on the outer surface of the bone, and the surgical controller 418 may then perform the registration procedure with the both original locations received and the additional locations received after the overlay process. The process repeats until the surgeon approves the registration. The specification now turns to intraoperative tunnel path planning.

### TUNNEL PATH PLANNING

Using the three-dimensional bone model an operative plan is preoperatively created that comprises a planned-tunnel path through the bone, including locations of the apertures into the bone that define the ends of the tunnel. Further, a depth of the planned-tunnel path is selected and a diameter of the tunnel path is selected.

In some embodiments, when planning a surgical solution, a surgeon may be presented (e.g., on the display device 414) with visual feedback of a region of exit-point placements that are achievable when placing the tunnel. For example, an overlay may be presented on an image of a bone and the overlay may include determined or calculated knee flexion angle, depicted as iso-knee flexion angle lines, to enable the surgeon to better approximate patient positioning.

In some cases, however, the surgeon may elect not to use planned-tunnel path, and thus elect not to use the preoperatively planned entry location, exit location, or both. Such an election can be based any of a number of reasons. For example, intraoperatively the surgeon may not be able to access the entry location for the planned-tunnel path, and thus may need to move the entry location to ensure sufficient access. As another example, during the intraoperative procedure the surgeon may determine that the planned tunnel entry location is misaligned with the attachment location of the native ACL to the femur. Further still, during the intraoperative procedure the surgeon may determine the tunnel entry location is too close to the posterior wall of the femur, increasing the likelihood of a bone chip sometimes referred to as a “back wall blowout.” Regardless of the reason for the election to change the tunnel path, in example systems the surgical controller 418 enables the surgeon to intraoperatively select a revised-tunnel entry, a revised-tunnel exit (if needed), and thus a revised-tunnel path through the bone.

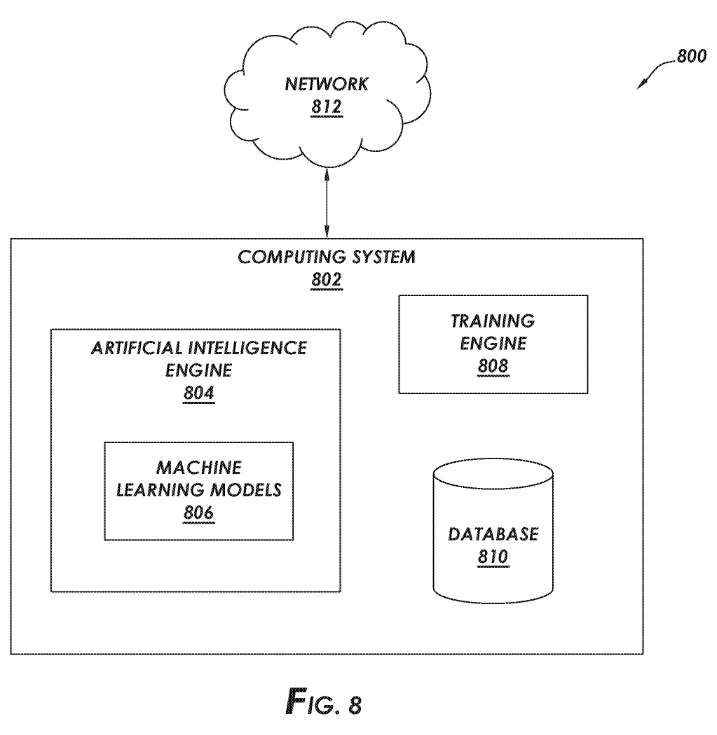

FIG. 7 is an example video display showing intraoperative changes to the tunnel path, in accordance with at least some embodiments. The display may be shown, for example, on the display device 414 (FIG. 4) associated with the device cart 402 (FIG. 4), or any other suitable location. In particular, FIG. 7 shows a portion of the bone in the video images as captured by the arthroscope 408 (FIG. 4) overlaid with the mesh model of the three-dimensional bone model and a preoperatively selected planned-tunnel path 700 comprising a planned-tunnel entry 702 and a planned-tunnel exit 704. However, for any number of reasons, the surgeon may elect to modify the tunnel entry location and/or the tunnel exit location, and thus modify the planned-tunnel path. Thus, the surgeon may provide to the surgical controller 418 (FIG. 4), and thus the surgical controller 418 may receive, a revised-tunnel entry location or just revised-tunnel entry 706. Providing the revised-tunnel entry 706 may comprise the surgeon touching the proposed location on the bone shown in the video images with a tracked tool, such as the touch probe 504 (FIG. 5) or the aimer 426 (FIG. 4). In some cases, the surgeon may select the revised-tunnel entry 706 based solely on what the surgeon sees of the bone shown in the video images. As a more specific example, the surgeon may select and provide the revised-tunnel entry 706 based on a location that can be reached by the aimer 426. In yet still other cases, the surgical controller 418 may generate a simulated fluoroscopic images from the three-dimensional bone model, and project thereon a Bernard & Hertel Quadrant or grid. The surgeon may then select the revised-tunnel entry 706 with the additional guidance provided by the Bernard & Hertel Quadrant.

### TUNNEL CREATION

With the revised-tunnel path 710 selected, the next step in the example method is creation of the actual tunnel. In most cases, creating the tunnel is a multistep process involving drilling an initial or pilot tunnel using a drill wire (e.g., drill wire 424 (FIG. 4)), and then using the drill wire as guide wire for one or more reamers to increase the diameter of the pilot tunnel to form the full-diameter actual tunnel through the bone. In some cases the actual tunnel has a counterbore associated with the intercondylar notch to accommodate the width of the autograft, and in such cases an additional reamer may be used to create the counterbore.

### SOFTWARE AND HARDWARE

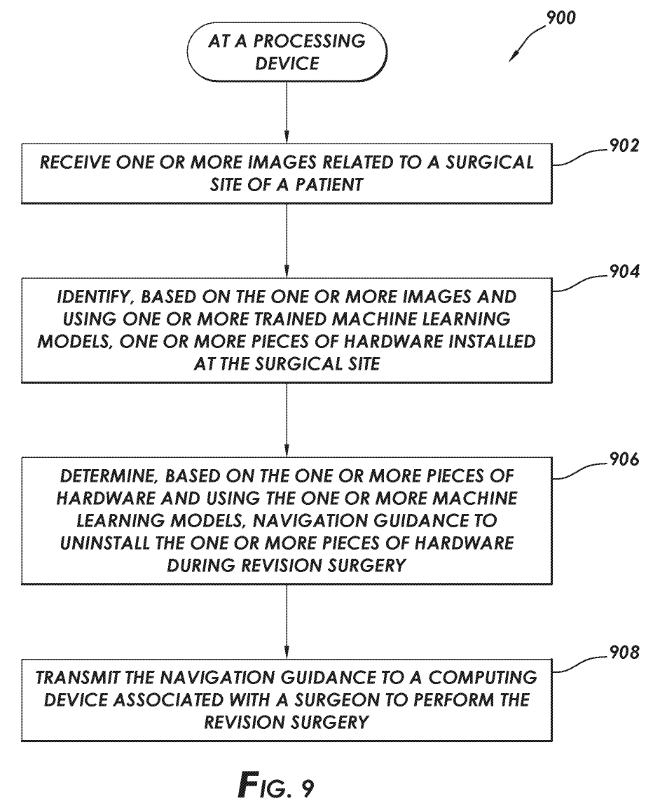

FIG. 8 illustrates a high-level component diagram of an illustrative system architecture 800 according to certain embodiments of this disclosure. In some embodiments, the system architecture 800 may include a computing system 802 communicatively coupled via a network 812. The computing system 802 may be a real-time software platform, include privacy software or protocols, or include security software or protocols. The computing system 802 may include one or more processing devices, memory devices, and/or network interface cards. The network interface cards may enable communication via a wireless protocol for transmitting data over short distances, such as Bluetooth, ZigBee, NFC, etc. Additionally, the network interface cards may enable communicating data via a wired protocol over short or long distances, and in one example, the computing system 802 may communicate with the network 812. Network 812 may be a public network (e.g., connected to the Internet via wired (Ethernet) or wireless (WiFi)), a private network (e.g., a local area network (LAN) or wide area network (WAN)), or a combination thereof. In some embodiments, network 812 may also comprise a node or nodes on the Internet of Things (IoT).

The computing system 802 may be any suitable computing device, such as an embedded computer device with display, a laptop, tablet, smartphone, or computer. The computing system 802 may include a display capable of presenting a user interface of an application. The application may be implemented in computer instructions stored on the one or more memory devices of the computing system 802 and executable by the one or more processing devices of the computing system 802.

The user interface may present various screens to a user that present various views including recommendations pertaining to performing revision surgery based on one or more probabilities of success or probabilities of collision that is detected based on one or more images using one or more machine learning models. The user interface may also present navigation guidance that includes an angle of approach for drilling a tunnel and/or inserting a tool to remove hardware included in a prior ACL reconstructive surgery and/or any suitable surgery. The user interface may also present an overlay that includes graphical elements indicating an insertion point for inserting a tool and/or drilling a tunnel, angle of approach for the tool and/or drill, information pertaining to a set of tools to be used to perform the revision surgery, step by step instructions how to remove the hardware installed in the person’s body, and the like. The computing system 802 may also include instructions stored on the one or more memories or memory devices that, when executed by the one or more processors or processing devices of the computing system 802, perform operations of any of the methods described herein.

In some embodiments, the computing system 802 may include one or more servers that form a distributed computing system, which may include a cloud computing system. The servers may be a rackmount server, a router, a personal computer, a portable digital assistant, a mobile phone, a laptop computer, a tablet computer, a camera, a video camera, a netbook, a desktop computer, a media center, any other device capable of functioning as a server, or any combination of the above. Each of the servers may include one or more processing devices, memory devices, data storage, or network interface cards. The servers may be in communication with one another via any suitable communication protocol.

The computing system 802 may execute an artificial intelligence engine 804 and one or more machine learning models 806, as described further herein. That is, the computing system 802 may execute an artificial intelligence (AI) engine 804 that uses one or more machine learning models 806 to perform at least one of the embodiments disclosed herein. The computing system 802 may also include a database 810 that stores data, knowledge, and data structures used to perform various embodiments. For example, the database 810 may store anatomical images, information pertaining to hardware used in reconstructive surgery, information pertaining to tools used to remove the hardware, information pertaining to revision surgery and removal of hardware, instructions related to revision surgery, and the like.

In some embodiments, the computing system 802 may include a training engine 808 capable of generating one or more machine learning models 806. Although depicted separately from the AI engine 804, the training engine 808 may, in some embodiments, be included in the AI engine 804 executing on the computing system 802. In some embodiments, the AI engine 804 may use the training engine 808 to generate the machine learning models 806 trained to perform inferencing and/or predicting operations. The machine learning models 806 may be trained to predict a probability of success of performing revision surgery, a probability of collision that is detected if revision surgery is performed, navigation guidance for performing the revision surgery, among other things. The one or more machine learning models 806 may be generated by the training engine 808 and may be implemented in computer instructions executable by one or more processing devices of the training engine 808. To generate the one or more machine learning models 806, the training engine 808 may train the one or more machine learning models 806. The one or more machine learning models 806 may be used by any of the methods described herein.

In some embodiments, the training engine 808 may be implemented in computer instructions stored on one or more memory devices and executed by one or more processing devices of the computing system 802. In some embodiments, the training engine 808 may be separate from the computing system 802 and may be a rackmount server, a router, a personal computer, a portable digital assistant, a smartphone, a laptop computer, a tablet computer, a netbook, a desktop computer, an Internet of Things (IoT) device, any other desired computing device, or any combination of the above. The training engine 808 may be cloud-based, be a real-time software platform, include privacy software or protocols, or include security software or protocols.

To generate the one or more machine learning models 806, the training engine 808 may train the one or more machine learning models 806. The training engine 808 may use a base training data set including inputs of labeled hardware data (e.g., images of hardware (bolts, screws, etc.) associated with surgery of a portion of a person’s body), among other things. One or more combinations of the inputs may be mapped to an output pertaining to one or more tools to use (e.g., including information related to a product, a diameter, a length, a guidewire, a screwdriver type, a head shape, a part number, a composition, etc.) to remove the hardware. In addition, the training data may include labeled inputs of images of a portion of a user’s body including installed hardware and/or a drilled tunnel for a performed reconstructive surgery and one or more outputs of navigation guidance to perform revision surgery. The navigation guidance may be based on the type of hardware identified in the one or more images of the portion of the patient’s body and/or the one or more tunnels identified in the one or more images. The navigation guidance may provide information related to one or more tools to use to remove the one or more identified pieces of hardware in the patient’s body, information related to an angle of approach for inserting the one or more tools to remove the one or more identified pieces of hardware, graphics for tools placement / insertion, graphics for entry points, step by step instructions for performing the revision surgery, and the like. In addition, the machine learning models 806 may be trained with training data including inputs of one or more images (CT images, X-ray images, etc.) of a portion of a patient’s body that experienced reconstructive surgery and outputs of a probability of success for performing revision surgery (e.g., removing hardware, drilling a new tunnel, etc.). Further, the machine learning models 806 may be trained with training data including inputs of one or more images of a portion of a patient’s body that experienced reconstructive surgery and outputs of a probability of colliding with installed hardware (e.g., screw) if revisions surgery is performed. In some examples, the machine learning models 806 may be trained to segment the bone and hardware in the images. The machine learning models 806 may identify the bones present in the image and identify the hardware present in the image by overlaying an identity / name of the bone and/or hardware.

The training engine 808 may use supervised learning in some embodiments to train the one or more machine learning models 806. Labeled data may be used by the training engine 808 to train the one or more machine learning models 806. In some examples, unsupervised learning may be used by the training engine 808 to train the one or more machine learning models 806. In some examples, reinforcement learning may be used by the training engine 808 to train the one or more machine learning models 806.

The training may include optimizing one or more parameters of the one or more machine learning models 806. For example, the parameters may include a number of hidden layers, a number of nodes used by each hidden layer, a weight, a type of activation function used, a type of loss function used, a batch size, a pooling size, number of iterations of training, etc.

The one or more machine learning models 806 may refer to model artifacts created by the training engine 808 using training data that includes training inputs and corresponding target outputs. The training engine 808 may find patterns in the training data wherein such patterns map the training input to the target output and generate the machine learning models 806 that capture these patterns. Although depicted as included in the computing system 802, in some embodiments, the artificial intelligence engine 804, the database 810, and/or the training engine 808 may reside on separate computing systems and/or servers.

The one or more machine learning models 806 may comprise, e.g., a single level of linear or non-linear operations (e.g., a support vector machine (SVM) or the machine learning models 806 may be a deep network, i.e., a machine learning model comprising multiple levels of non-linear operations. Examples of deep networks are neural networks, including generative adversarial networks, convolutional neural networks, recurrent neural networks with one or more hidden layers, and fully connected neural networks (e.g., each artificial neuron may transmit its output signal to the input of the remaining neurons, as well as to itself). For example, the machine learning model may include numerous layers or hidden layers that perform calculations (e.g., dot products) using various neurons. In some embodiments, the one or more machine learning models 806 may be trained via supervised learning, unsupervised learning, and/or reinforcement learning.

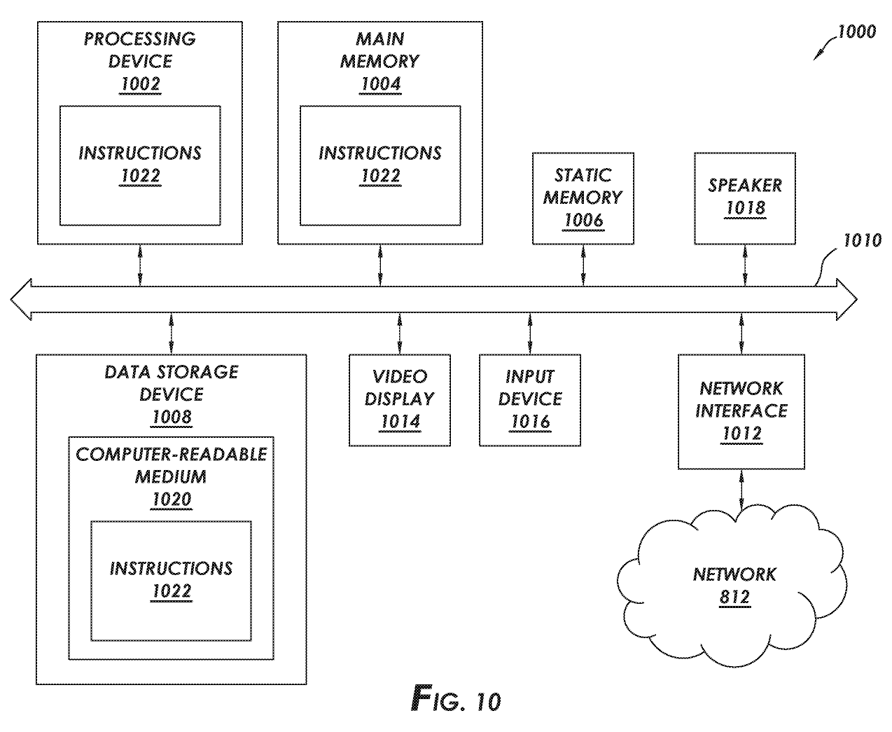

FIG. 9 is an example flow chart of a method 900 for using machine learning models to provide navigation guidance for revision surgery, in accordance with at least some embodiments. The method 900 may be performed by one or more processors or processing devices, such as included in the surgical controller 418 and/or computing system 802, for example. The method may be implemented in computer instructions stored in one or more memories that are executed by the one or more processors.

At block 902, the processor may receive one or more images related to a surgical site of a patient. The one or more images may be received from an arthroscope. The one or more images may be computed tomography images including details of a portion of a patient’s body. The one or more images may include a patient’s bones, tissue, and/or hardware (e.g., screws, bolts, nails, plates, etc.) that was installed during reconstructive surgery.

At block 904, the processor may identify, based on the one or more images and using one or more trained machine learning models, one or more pieces of hardware installed at the surgical site. The one or more machine learning models may be trained to identify the hardware installed at the surgical site and may output a type of hardware, a manufacturer of the hardware, a length of the hardware, a diameter of the hardware, a type of tool used to install / uninstall the hardware, a guidewire, a head shape of the hardware, a composition of the hardware, or some combination thereof. The machine learning models may be trained to identify an angle of installation of the one or more pieces of hardware, a tunnel orientation in the portion of the patient’s body, an entry point for revision surgery, a thickness of a bone wall, etc.

At block 906, the processor may determine, based on the one or more pieces of hardware and using the one or more machine learning models, navigation guidance to uninstall the one or more pieces of hardware during revision surgery. In some examples, the navigation guidance may include at least an angle of approach to uninstall the one or more pieces of hardware. In some examples, the navigation guidance may include an overlay to present on a display. The overlay may include one or more graphical elements related to the navigation guidance. The overlay may be presented concurrently over the one or more images of the portion of the patient’s body and may be presented in real-time with step by step instructions of how to perform the revision surgery as the surgeon is operating on the patient. Further, the display may present alerts if the surgeon’s tool deviates from the recommended navigation guidance by more than a threshold amount. The alert may indicate that the tool is going to collide with an anatomical feature of the patient and/or a piece of hardware installed in the portion of the patient’s body. The navigation guidance may be presented in real-time such that the surgeon is enabled to correctly perform the revision surgery.

At block 908, the processor may transmit the navigation guidance to a computing device associate with a surgeon to perform the revision surgery. The navigation guidance may be presented via a display, such as display device 414 and/or a user interface of the computing system 802.The navigation guidance may indicate which tool to use at each step of the surgery and may show the surgeon how to insert a tool, how to remove certain pieces of hardware (identified by the machine learning models), etc.

In some examples, the processor may determine, using the one or more machine learning models, a probability of success of uninstalling the one or more pieces of hardware during the revision surgery. In some examples, the processor may determine, based on the probability, the navigation guidance to uninstall the one or more pieces of hardware during the revision surgery. In addition, in some examples, the processor may determine a probability of collision with previously installed hardware and/or a previously drilled tunnel if the surgeon is to perform the revision surgery.

In some embodiments, the processor may transmit, based on the probability, one or more recommendations pertaining to the revision surgery. For example, if the probability of success satisfies a threshold probability (e.g., greater than 95 percent probability of success), then the recommendation may recommend that the revision surgery be performed. The probability of success may be determined based on comparing a corpus of data of similar images of reconstructive surgeries that have similar anatomical features and similarly used hardware. The corpus of data may indicate whether revision surgery was successfully performed on the similarly situated patients or whether it was not successfully performed. Further, the recommendation may include instructions on a certain angle of approach to uninstall the one or more identified pieces of hardware. The angle of approach may be based on the angle at which the one or more pieces of hardware are installed in one or more anatomical features of the patient, one or more types of hardware, one or more manufacturers of the hardware, the orientation of the tunnel that was drilled, etc. Further, the recommendation may include instructions specifying one or more tools to use to uninstall the one or more identified pieces of hardware.

In some examples, if the probability of success does not satisfy the threshold probability (e.g., less than 95 percent probability of success), then the recommendation may not recommend proceeding with performing the revision surgery.

In some examples, the processor may determine, using the one or more trained machine learning models, one or more tools to use to uninstall the one or more pieces of hardware during the revision surgery. For example, the machine learning models may be trained based on a corpus of data that matches pieces of hardware with associated with tools used to install / uninstall those pieces of hardware.

FIG. 10 shows an example computer system 1000. In one example, computer system 1000 may correspond to the surgical controller 418, the computing system 802, a tablet device within the surgical room, or any other system that implements any or all the various methods discussed in this specification. The computer system 1000 may be connected (e.g., networked) to other computer systems in a local-area network (LAN), an intranet, and/or an extranet (e.g., device cart 402 network), or at certain times the Internet (e.g., when not in use in a surgical procedure). The computer system 1000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1000 includes a processing device 1002, a main memory 1004 (e.g., read-only memory (ROM), flash memory, dynamic random access memory (DRAM) such as synchronous DRAM (SDRAM)), a static memory 1006 (e.g., flash memory, static random access memory (SRAM)), and a data storage device 1008, which communicate with each other via a bus 1010.

Processing device 1002 represents one or more general-purpose processing devices such as a microprocessor, central processing unit, or the like. More particularly, the processing device 1002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1002 may also be one or more special-purpose processing devices such as an application specific integrated circuit (ASIC), a field programmable gate array (FPGA), a digital signal processor (DSP), network processor, or the like. The processing device 1002 is configured to execute instructions for performing any of the operations and steps discussed herein. Once programmed with specific instructions, the processing device 1002, and thus the entire computer system 1000, becomes a special-purpose device, such as the surgical controller 418 and/or computing system 802.

The computer system 1000 may further include a network interface device 1012 for communicating with any suitable network 812 (e.g., the device cart 402 network). The computer system 1000 also may include a video display 1014 (e.g., display device 414), one or more input devices 1016 (e.g., a microphone, a keyboard, and/or a mouse), and one or more speakers 1018. In one illustrative example, the video display 1014 and the input device(s) 1016 may be combined into a single component or device (e.g., an LCD touch screen).

The data storage device 1008 may include a computer-readable storage medium 1020 on which the instructions 1022 (e.g., implementing any methods and any functions performed by any device and/or component depicted described herein) embodying any one or more of the methodologies or functions described herein is stored. The instructions 1022 may also reside, completely or at least partially, within the main memory 1004 and/or within the processing device 1002 during execution thereof by the computer system 1000. As such, the main memory 1004 and the processing device 1002 also constitute computer-readable media. In certain cases, the instructions 1022 may further be transmitted or received over a network via the network interface device 1012.

While the computer-readable storage medium 1020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

### Clauses:

1. A computer-implemented method comprising:

receiving one or more images related to a surgical site of a patient;

identifying, based on the one or more images and using one or more trained machine learning models, one or more pieces of hardware installed at the surgical site;

determining, based on the one or more pieces of hardware and using the one or more machine learning models, navigation guidance to uninstall the one or more pieces of hardware during revision surgery; and

transmitting the navigation guidance to a computing device associated with a surgeon to perform the revision surgery.

2. The computer-implemented method of any clause herein, further comprising:

determining, using the one or more machine learning models, a probability of success of uninstalling the one or more pieces of hardware during the revision surgery.

3. The computer-implemented method of any clause herein, further comprising:

determining, based on the probability, the navigation guidance to uninstall the one or more pieces of hardware during the revision surgery.

4. The computer-implemented method of any clause herein, further comprising:

transmitting, based on the probability, one or more recommendations pertaining to the revision surgery.

5. The computer-implemented method of any clause herein, further comprising:

determining, using the one or more trained machine learning models, one or more tools to use to uninstall the one or more pieces of hardware during the revision surgery.

6. The computer-implemented method of any clause herein, wherein the navigation guidance comprises at least an angle of approach to uninstall the one or more pieces of hardware.

7. The computer-implemented method of any clause herein, wherein the navigation guidance comprises an overlay to present on a display of the computing device, and the overlay includes one or more graphical elements related to the navigation guidance.

8. A system comprising:

a memory storing instructions; and

a processing device communicatively coupled to the memory, wherein the processing device executes the instructions to:

receive one or more images related to a surgical site of a patient;

identify, based on the one or more images and using one or more trained machine learning models, one or more pieces of hardware installed at the surgical site;

determine, based on the one or more pieces of hardware and using the one or more machine learning models, navigation guidance to uninstall the one or more pieces of hardware during revision surgery; and

transmit the navigation guidance to a computing device associated with a surgeon to perform the revision surgery.

9. The system of any clause herein, wherein the processing device is further to:

determine, using the one or more machine learning models, a probability of success of uninstalling the one or more pieces of hardware during the revision surgery.

10. The system of any clause herein, wherein the processing device is further to:

determine, based on the probability, the navigation guidance to uninstall the one or more pieces of hardware during the revision surgery.

11. The system of any clause herein, wherein the processing device is further to:

transmit, based on the probability, one or more recommendations pertaining to the revision surgery.

12. The system of any clause herein, wherein the processing device is further to:

determine, using the one or more machine learning models, one or more tools to use to uninstall the one or more pieces of hardware during the revision surgery.

13. The system of any clause herein, wherein the navigation guidance comprises at least an angle of approach to uninstall the one or more pieces of hardware.

14. The system of any clause herein, wherein the navigation guidance comprises an overlay to present on a display of the computing device, and the overlay includes one or more graphical elements related to the navigation guidance.

15. A tangible, non-transitory computer-readable medium storing instructions that, when executed, cause a processing device to:

receive one or more images related to a surgical site of a patient;

identify, based on the one or more images and using one or more trained machine learning models, one or more pieces of hardware installed at the surgical site;

determine, based on the one or more pieces of hardware and using the one or more machine learning models, navigation guidance to uninstall the one or more pieces of hardware during revision surgery; and

transmit the navigation guidance to a computing device associated with a surgeon to perform the revision surgery.

16. The computer-readable medium of any clause herein, wherein the processing device is further to:

determine, using the one or more machine learning models, a probability of success of uninstalling the one or more pieces of hardware during the revision surgery.

17. The computer-readable medium of any clause herein, wherein the processing device is further to:

determine, based on the probability, the navigation guidance to uninstall the one or more pieces of hardware during the revision surgery.

18. The computer-readable medium of any clause herein, wherein the processing device is further to:

transmit, based on the probability, one or more recommendations pertaining to the revision surgery.

19. The computer-readable medium of any clause herein, wherein the processing device is further to:

determine, using the one or more machine learning models, one or more tools to use to uninstall the one or more pieces of hardware during the revision surgery.

20. The computer-readable medium of any clause herein, wherein the navigation guidance comprises an overlay to present on a display of the computing device, and the overlay includes one or more graphical elements related to the navigation guidance.

What is claimed is:

1. A computer-implemented method comprising:
receiving one or more images related to a surgical site of a patient;
identifying, based on the one or more images and using one or more trained machine learning models, one or more pieces of hardware installed at the surgical site;
determining, based on the one or more pieces of hardware and using the one or more machine learning models, navigation guidance to uninstall the one or more pieces of hardware during revision surgery; and
transmitting the navigation guidance to a computing device associated with a surgeon to perform the revision surgery.

2. The computer-implemented method of claim 1, further comprising:
determining, using the one or more machine learning models, a probability of success of uninstalling the one or more pieces of hardware during the revision surgery.

3. The computer-implemented method of claim 2, further comprising:
determining, based on the probability, the navigation guidance to uninstall the one or more pieces of hardware during the revision surgery.

4. The computer-implemented method of claim 2, further comprising:
transmitting, based on the probability, one or more recommendations pertaining to the revision surgery.

5. The computer-implemented method of claim 1, further comprising:
determining, using the one or more trained machine learning models, one or more tools to use to uninstall the one or more pieces of hardware during the revision surgery.

6. The computer-implemented method of claim 1, wherein the navigation guidance comprises at least an angle of approach to uninstall the one or more pieces of hardware.

7. The computer-implemented method of claim 1, wherein the navigation guidance comprises an overlay to present on a display of the computing device, and the overlay includes one or more graphical elements related to the navigation guidance.

8. A system comprising:
a memory storing instructions; and
a processing device communicatively coupled to the memory, wherein the processing device executes the instructions to:
receive one or more images related to a surgical site of a patient;
identify, based on the one or more images and using one or more trained machine learning models, one or more pieces of hardware installed at the surgical site;
determine, based on the one or more pieces of hardware and using the one or more machine learning models, navigation guidance to uninstall the one or more pieces of hardware during revision surgery; and
transmit the navigation guidance to a computing device associated with a surgeon to perform the revision surgery.

9. The system of claim 8, wherein the processing device is further to:
determine, using the one or more machine learning models, a probability of success of uninstalling the one or more pieces of hardware during the revision surgery.

10. The system of claim 9, wherein the processing device is further to:
determine, based on the probability, the navigation guidance to uninstall the one or more pieces of hardware during the revision surgery.

11. The system of claim 9, wherein the processing device is further to:
transmit, based on the probability, one or more recommendations pertaining to the revision surgery.

12. The system of claim 8, wherein the processing device is further to:
determine, using the one or more machine learning models, one or more tools to use to uninstall the one or more pieces of hardware during the revision surgery.

13. The system of claim 8, wherein the navigation guidance comprises at least an angle of approach to uninstall the one or more pieces of hardware.

14. The system of claim 8, wherein the navigation guidance comprises an overlay to present on a display of the computing device, and the overlay includes one or more graphical elements related to the navigation guidance.

15. A tangible, non-transitory computer-readable medium storing instructions that, when executed, cause a processing device to:
receive one or more images related to a surgical site of a patient;
identify, based on the one or more images and using one or more trained machine learning models, one or more pieces of hardware installed at the surgical site;
determine, based on the one or more pieces of hardware and using the one or more machine learning models, navigation guidance to uninstall the one or more pieces of hardware during revision surgery; and
transmit the navigation guidance to a computing device associated with a surgeon to perform the revision surgery.

16. The computer-readable medium of claim 15, wherein the processing device is further to:
determine, using the one or more machine learning models, a probability of success of uninstalling the one or more pieces of hardware during the revision surgery.

17. The computer-readable medium of claim 16, wherein the processing device is further to:
determine, based on the probability, the navigation guidance to uninstall the one or more pieces of hardware during the revision surgery.

18. The computer-readable medium of claim 16, wherein the processing device is further to:
transmit, based on the probability, one or more recommendations pertaining to the revision surgery.

19. The computer-readable medium of claim 15, wherein the processing device is further to:
determine, using the one or more machine learning models, one or more tools to use to uninstall the one or more pieces of hardware during the revision surgery.

20. The computer-readable medium of claim 15, wherein the navigation guidance comprises an overlay to present on a display of the computing device, and the overlay includes one or more graphical elements related to the navigation guidance.
